(19) United States
(12) Patent Application Publication
(10) Pub. No.: US 2008/0021457 A1
(43) Pub. Date: Jan. 24, 2008

(54) Zygapophysial joint repair system

(75) Inventors: Kent M. Anderson, Memphis, TN (US); Aurelien Bruneau, Memphis, TN (US); Eric C. Lange, Collierville, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: Warsaw Orthopedic Inc., Warsaw, IN (US)

(21) Appl. No.: 11/481,079

(22) Filed: Jul. 5, 2006

(51) Int. Cl. A61F 2/30

(52) U.S. Cl. 606/61

(57) ABSTRACT

A kit includes a mesh material adapted to engage a zygapophysial joint, and includes an indicator associating the kit with treatment of the zygapophysial joint.

### FIELD OF THE DISCLOSURE

This disclosure, in general, relates to repair or support systems for use in connection with a zygapophysial joint.

### BACKGROUND

In human anatomy, the spine is a generally flexible column that can withstand tensile and compressive loads. The spine also allows bending motion and provides a place of attachment for keels, muscles, and ligaments. Generally, the spine is divided into four sections: the cervical spine, the thoracic or dorsal spine, the lumbar spine, and the pelvic spine. The pelvic spine generally includes the sacrum and the coccyx. The sections of the spine are made up of individual bones called vertebrae. Three joints reside between each set of two vertebrae: a larger intervertebral disc between the two vertebral bodies and two zygapophysial joints located posterolaterally relative to the vertebral bodies and between opposing articular processes.

The intervertebral discs generally function as shock absorbers and as joints. Further, the intervertebral discs can absorb the compressive and tensile loads to which the spinal column can be subjected. At the same time, the intervertebral discs can allow adjacent vertebral bodies to move relative to each other, particularly during bending or flexure of the spine. Thus, the intervertebral discs are under constant muscular and gravitational pressure and generally, the intervertebral discs are the first parts of the lumbar spine to show signs of deterioration.

The zygapophysial joints permit movement in the vertical direction, while limiting rotational motion of the two adjoining vertebrae. In addition, capsular ligaments surround the zygapophysial joints, discouraging excess extension and torsion. In addition to intervertebral disc degradation, zygapophysial joint degeneration is also common because the zygapophysial joints are in almost constant motion with the spine. In fact, zygapophysial joint degeneration and disc degeneration frequently occur together. Generally, although one can be the primary problem while the other is a secondary problem resulting from the altered mechanics of the spine, by the time surgical options are considered, both zygapophysial joint degeneration and disc degeneration typically have occurred. For example, the altered mechanics of the zygapophysial joints or intervertebral disc can cause spinal stenosis, degenerative spondylolisthesis, and degenerative scoliosis.

Furthermore, acute strenuous events, such as whiplash or overextension, can damage capsular ligaments. Such damage to capsular ligaments if untreated can lead to degradation of the zygapophysial joint.

### BRIEF DESCRIPTION OF THE DRAWINGS

The present disclosure may be better understood, and its numerous features and advantages made apparent to those skilled in the art by referencing the accompanying drawings.

FIG. 1 includes a lateral view of a portion of a vertebral column.

FIG. 2 includes a lateral view of a pair of adjacent vertebrae.

FIG. 3 includes a top plan view of a vertebra.

FIG. 4 includes a cross-sectional view of two adjacent vertebrae.

FIG. 5 and FIG. 6 include illustrations of exemplary embodiments of joint repair or support systems.

FIG. 7, FIG. 8, FIG. 9, FIG. 10, FIG. 11, and FIG. 12 include illustrations of exemplary mesh material.

FIG. 13, FIG. 14, and FIG. 15 include illustrations of exemplary strands.

FIG. 16 and FIG. 17 include illustrations of exemplary joint support or repair systems.

FIG. 18 includes an illustration of an exemplary zygapophysial joint.

FIG. 19 includes a flow diagram illustrating an exemplary method to treat a patient.

FIG. 20 includes an illustration of an exemplary kit including a joint repair or support system.

### DESCRIPTION OF THE EMBODIMENTS

In a particular embodiment, a kit includes a mesh material or a strand material and includes an indicator or instructions. The mesh material or the strand material are adapted to wrap around a zygapophysial joint. In addition, the kit can include a fastener to secure the mesh material or the strand material. For example, the fastener can be adapted to secure the mesh material to a process associated with the zygapophysial joint. In another example, the fastener can be adapted to secure the strand material to itself.

In an exemplary embodiment, a kit includes a mesh material adapted to engage a zygapophysial joint, and includes an indicator associating the kit with treatment of the zygapophysial joint.

In another exemplary embodiment, a device includes a mesh material adapted to engage a zygapophysial joint, and includes a fastener configured to secure the mesh material to an articular process associated with the zygapophysial joint.

In a further exemplary embodiment, a kit includes a strand material configured to engage a zygapophysial joint, and includes instructions indicating that the strand material is to be encircled around two articular processes associated with the zygapophysial joint.

In an additional exemplary embodiment, a method to treat a zygapophysial joint includes wrapping a mesh material around a zygapophysial joint, securing the mesh material to a first articular process associated with the zygapophysial joint, and securing the mesh material to a second articular process associated with the zygapophysial joint.

In a further exemplary embodiment, a method to treat a zygapophysial joint includes wrapping a strand material around a zygapophysial joint, and securing the strand material to engage a first and a second articular process associated with the zygapophysial joint.

### Description of Relevant Anatomy

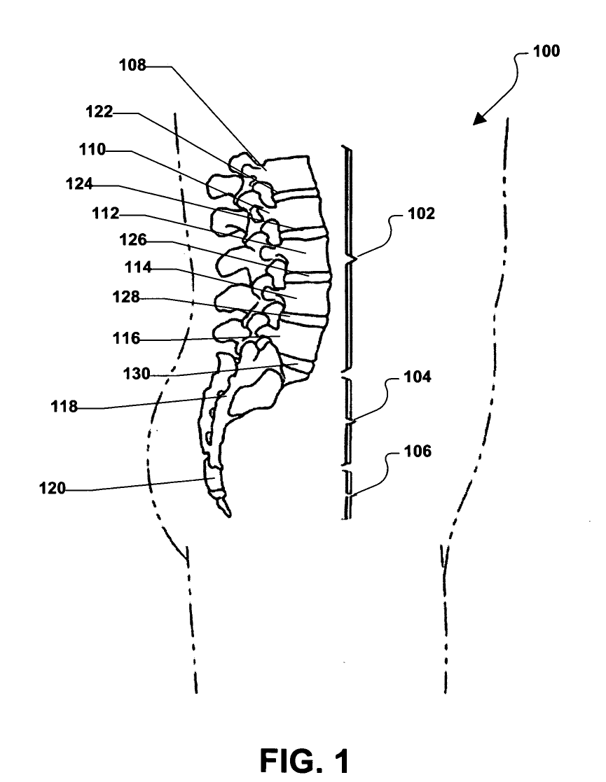

Referring initially to FIG. 1, a portion of a vertebral column, designated 100, is shown. As depicted, the vertebral column 100 includes a lumbar region 102, a sacral region 104, and a coccygeal region 106. The vertebral column 100 also includes a cervical region and a thoracic region. For clarity and ease of discussion, the cervical region and the thoracic region are not illustrated.

As illustrated in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118. In addition, two zygapophysial joints, often referred to as facet joints, are located between each adjacent pair of vertebrae.

In a particular embodiment, if one of the intervertebral lumbar discs 122, 124, 126, 128, 130 is diseased, degenerated, or damaged, or if one of the zygapophysial joints is diseased, degenerated or damaged, that disc or joint can be treated with a therapeutic agent or replaced with a prosthetic device.

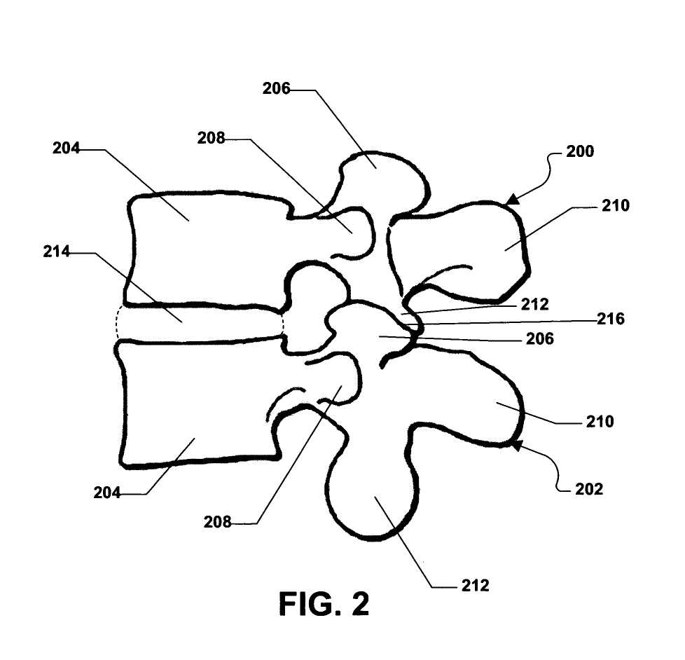

FIG. 2 depicts a detailed lateral view of two adjacent vertebrae, e.g., two of the lumbar vertebrae 108, 110, 112, 114, 116 illustrated in FIG. 1. FIG. 2 illustrates a superior vertebra 200 and an inferior vertebra 202. As illustrated, each vertebra 200, 202 includes a vertebral body 204, a superior articular process 206, a transverse process 208, a spinous process 210 and an inferior articular process 212. FIG. 2 further depicts an intervertebral disc 214 between the superior vertebra 200 and the inferior vertebra 202. A zygapophysial joint 216 is located between the inferior articular process 212 of the superior vertebra 200 and the superior articular process 206 of the inferior vertebra 202. As described in greater detail below, a repair or support device according to one or more of the embodiments described herein can be installed around the zygapophysial joint 216 and the articular processes 206 and 212.

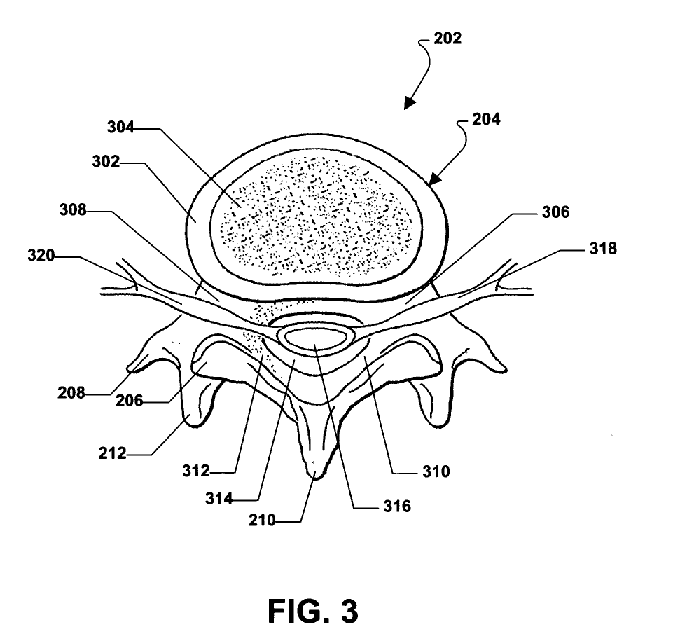

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

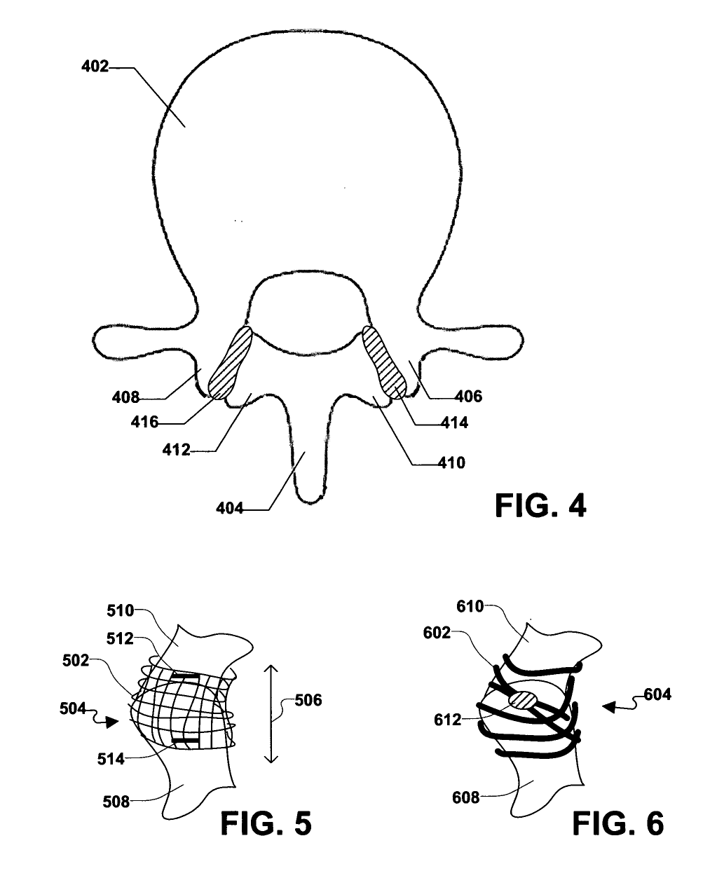

FIG. 4 includes a cross-sectional view of the spine illustrating a portion of a superior vertebra 404 and a portion of an inferior vertebra 402. The inferior vertebra 402 includes superior articular processes 406 and 408 and the superior vertebra 404 includes inferior articular processes 410 and 412. Between the superior articular process 406 and the inferior articular process 410 is a zygapophysial joint 414 and between the superior articular process 408 and the inferior articular process 412 is a zygapophysial joint 416.

When damaged or degraded, the zygapophysial joints 414 and 416 can be treated. For example, the zygapophysial joints 414 and 416 can be replaced, partially or wholly, with an implant. Such implants can be configured to fuse the inferior articular process (406 or 408) to the superior articular process (410 or 412). Alternatively, such implants can act to mimic the tissue of the zygapophysial joints 414 and 416. In another example, the zygapophysial joints 414 and 416 can be treated with therapeutic agents injected into the zygapophysial joints 414 and 416.

### Description of Embodiments of the Device

In an exemplary embodiment, the zygapophysial joint can be wrapped with a mesh material or a strand material. For example, a mesh material can be wrapped around the zygapophysial joint and secured to the inferior or the superior articular processes associated with the zygapophysial joint. In another example, a strand material can be wrapped around the articular processes associated with the zygapophysial joint and secured to itself.

FIG. 5 includes an illustration of an exemplary mesh material 502 wrapped around a zygapophysial joint 504. The zygapophysial joint 504 is formed between a superior articular process 508 of an inferior vertebra and an inferior articular process 510 of a superior vertebra. The directional indicator 506 indicates the general axis of the spine formed by the vertebra of which the processes 508 and 510 are part. As illustrated, the mesh material 502 is secured to the inferior articular process 510 via a fastener 512 and to the superior articular process 508 via a fastener 514. Alternatively, the mesh material 502 can be secured to itself via the fastener 512 or the fastener 514.

In another exemplary embodiment, FIG. 6 includes an illustration of an exemplary strand material 602 wrapped around a zygapophysial joint 604. The zygapophysial joint 604 is formed between a superior articular process 608 of an inferior vertebra and an inferior articular process 610 of a superior vertebra. The strand material 602 can engage the inferior articular process 610 and the superior articular process 608 by being wrapped around the processes 608 and 610. The strand material 602 can be secured to itself by the fastener 612.

In general, the mesh material or the strand material can be surgically installed in a patient by a healthcare practitioner. In an exemplary embodiment, the mesh material or the strand material can be included in a kit with an indicator as to the use of the mesh material or the strand material. For example, the indicator can include contraindications as to the use of the materials or can include instructions relating to how the materials are to be prepared or implanted.

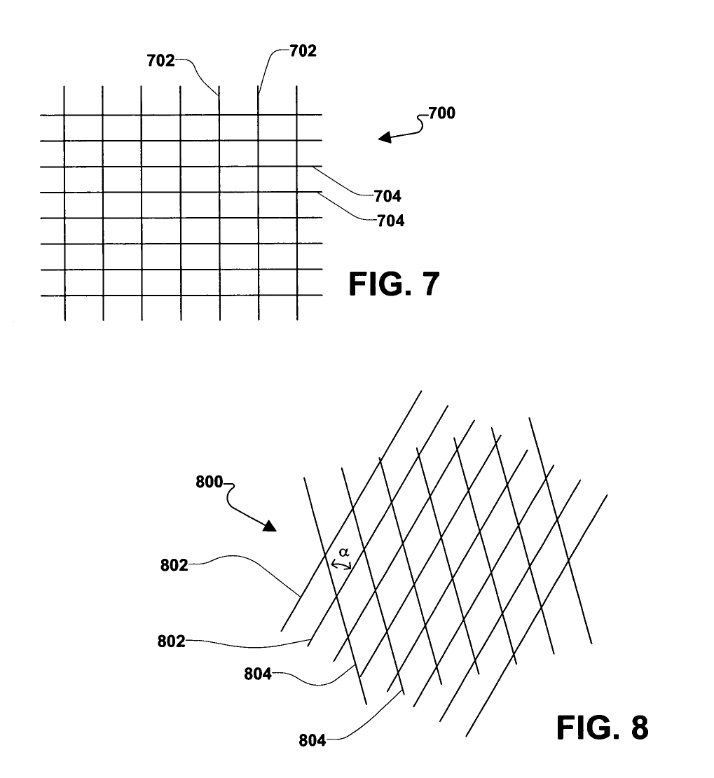

In an exemplary embodiment, the mesh material can include a sheet of strands interwoven together or secured together with a coating. FIG. 7 includes an illustration of an exemplary mesh material 700 including interwoven strands 702 and 704. In an example, the interwoven strand 702 represents a warp strand and the interwoven strand 704 represents a weft strand.

The strands 702 and 704 can be formed of a metallic material, a polymeric material, or any combination thereof. An exemplary polymeric material can include polyester, polypropylene, polyethylene, halogenated polyolefin, fluoropolyolefin, polybutadiene, polysulfone, polyaryletherketone, polyurethane or copolymers thereof, silicone, polyimide, polyamide, polyetherimide, a hydrogel, or any combination thereof. An exemplary polyaryletherketone (PAEK) material can include polyetherketone (PEK), polyetheretherketone (PEEK), polyetherketoneketone (PEKK), polyetherketoneetherketoneketone (PEKEKK), or any combination thereof. An exemplary silicone can include dialkyl silicones, fluorosilicones, or any combination thereof. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP) polylactic acid (PLA), or any combination thereof. An exemplary metallic material includes stainless steel, titanium, platinum, tantalum, gold or their alloys, as well as gold-plated ferrous alloy, platinum-plated ferrous alloy, cobalt-chromium alloy, titanium nitride coated stainless steel, or any combination thereof. In a particular embodiment, the strands 702 and 704 can be formed of resorbable material that can gradually be absorbed by the patient's body. The strands 702 and 704 can be formed of the same material. Alternatively, the strands 702 can be formed of a different material than the strands 704. In various exemplary embodiments, one or more of the strands can be formed of a biostable material, which is not substantially bioresorbable or biodegradable.

In the example illustrated in FIG. 7, the warp strands 702 and the weft strands 704 form a substantially orthogonal pattern, forming approximately 90° angles at the intersection between the warp strands 702 and the weft strands 704. When installed in a patient, the mesh material can be positioned such that the warp strands 702 align with the general axis of an upright spine. In another example, the weft strand 704 can align with the general axis.

In an alternative embodiment, the strands can intersect to form acute angles. For example, FIG. 8 includes an illustration of an interwoven mesh material 800 including warp strands 802 and weft strands 804 that intersect to form an acute angle α. In an exemplary embodiment, the acute angle α can be not greater than about 65°, such as not greater than about 45°. In a particular example, the mesh material can be installed such that a bisection of the angle α can align with the general axis. Alternatively, the warp strands 802 or the weft strands 804 can be aligned with the general axis.

In a particular embodiment, the alignment of the strands and the angle formed by the intersection of the strands can influence the performance of the mesh material. For example, alignment of the strands and the angle formed by the strands can affect the relative motion of the articular processes permitted by the mesh material.

In a further exemplary embodiment, the mesh material can include a therapeutic agent. In an example, the therapeutic agent can be included in a controlled release material incorporated into the mesh material. In another example, the mesh material can be configured to enclose the therapeutic agent, holding the agent in proximity to a desired location. In a further example, the mesh material can be coated in a release material.

In an example illustrated in FIG. 9, the mesh material 900 can include interwoven strands that include therapeutic agent. For example, the mesh material 900 can include warp strands 902 and weft strands 906 forming an interwoven material. In an example, a therapeutic strand 904 can be included between the warp strands 902. In another example, a therapeutic strand 908 can be included between the weft strands 906. In an exemplary embodiment, the therapeutic strands (904 and 908) can be formed of controlled release materials. For example, the therapeutic strands (904 and 908) can be formed of hydrogel materials. In another example, the therapeutic strands (904 and 908) can include a coating including the therapeutic agent. For example, the coating can include a slow dissolving solid matrix that releases the therapeutic agent as it dissolves.

An exemplary therapeutic agent includes a growth factor. The growth factor can be generally suited to promote the formation of tissues, especially of the type(s) naturally occurring as components of a zygapophysial joint. For example, the growth factor can promote the growth or viability of tissue or cell types occurring in the zygapophysial joint, such as chondrocytes, as well as space filling cells, such as fibroblasts, or connective tissue cells, such as ligament or tendon cells. Alternatively or in addition, the growth factor can promote the growth or viability of tissue types occurring around the zygapophysial joint, as well as space filling cells, such as fibroblasts, or connective tissue cells, such as ligament or tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

In another example, the therapeutic agent can include a soluble tumor necrosis factor α-receptor, a pegylated soluble tumor necrosis factor α-receptor, a monoclonal antibody, a polyclonal antibody, an antibody fragment, a COX-2 inhibitor, a metalloprotease inhibitor, a glutamate antagonist, a glial cell derived neurotrophic factor, a B2 receptor antagonist, a substance P receptor (NK1) antagonist, a downstream regulatory element antagonistic modulator (DREAM), iNOS, an inhibitor of tetrodotoxin (TTX)-resistant Na+-channel receptor subtypes PN3 and SNS2, an inhibitor of interleukin, a TNF binding protein, a dominant-negative TNF variant, Nanobodies™, a kinase inhibitor, or any combination thereof. Another exemplary therapeutic agent can include Adalimumab, Infliximab, Etanercept, Pegsunercept (PEG sTNF-R1), Onercept, Kineret®, sTNF-R1, CDP-870, CDP-571, CNI-1493, RDP58, ISIS 104838, 1→3-β-D-glucan, Lenercept, PEG-sTNFRII Fc Mutein, D2E7, Afelimomab, AMG 108, 6-methoxy-2-napthylacetic acid or betamethasone, capsaiein, civanide, TNFRc, ISIS2302 and GI 129471, integrin antagonist, alpha-4 beta-7 integrin antagonist, cell adhesion inhibitor, interferon gamma antagonist, CTLA4-Ig agonist/antagonist (BMS-188667), CD40 ligand antagonist, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL 15 antibody), or any combination thereof.

In another exemplary embodiment, the therapeutic agent can be included in a gel, absorbent material, or dissolvable matrix included in the mesh material adjacent a woven material. FIG. 10 includes an illustration of an exemplary mesh material 1000 that can include a woven material 1002 and a release material 1004 adjacent the woven material. In a further example, the mesh material 1000 can include a second woven material 1006 on an opposite side of the release material 1004 from the first woven material 1002. As illustrated in FIG. 10, the release material 1004 can be between the first and second woven materials (1002 and 1006). In an alternative embodiment, the release material 1004 can be on an inside surface of the woven material 1002 relative to the zygapophysial joint surrounded by the mesh material 1000 (i.e., the release material 1004 can be on a side of the woven material 1002 closest to the zygapophysial joint).

In an alternative example, the therapeutic agent can be injected directly into the zygapophysial joint that is surrounded by the mesh material. In another example, the therapeutic agent can be included in a gel or solid matrix that is injected into a space between the zygapophysial joint and the mesh material surrounding the zygapophysial joint.

In a further exemplary embodiment, the mesh material can include adjacent strands that are spaced apart. Alternatively, adjacent parallel strands can be interwoven in a tight pattern, close together. For example, FIG. 11 illustrates an exemplary mesh material 1100 that includes warp strands 1102 that are spaced apart and weft strands 1104 that are spaced apart. The spacing of the strands 1102 and 1104 effect open areas 1106. In the illustrated woven material 1100, the open areas 1106 form at least about 75% of the total area of a surface of the woven material 1100. In an alternative example, the strands are interwoven close together. For example, FIG. 12 includes an illustration of a mesh material including a woven sheet having open areas 1202 that form not greater than about 20% of the total area of the surface of the woven material. In particular, the open areas 1202 can form not greater than about 5% of the total surface area.

The strands forming the mesh material or the strand material can be single fiber strands or fibrous strands. For example, metal strands and extruded polymer strands are typically single fiber strands. Alternatively, polymer fibers can be intertwined to form a strand. The fibrous strands can be randomly intertwined or structured intertwined fibers.

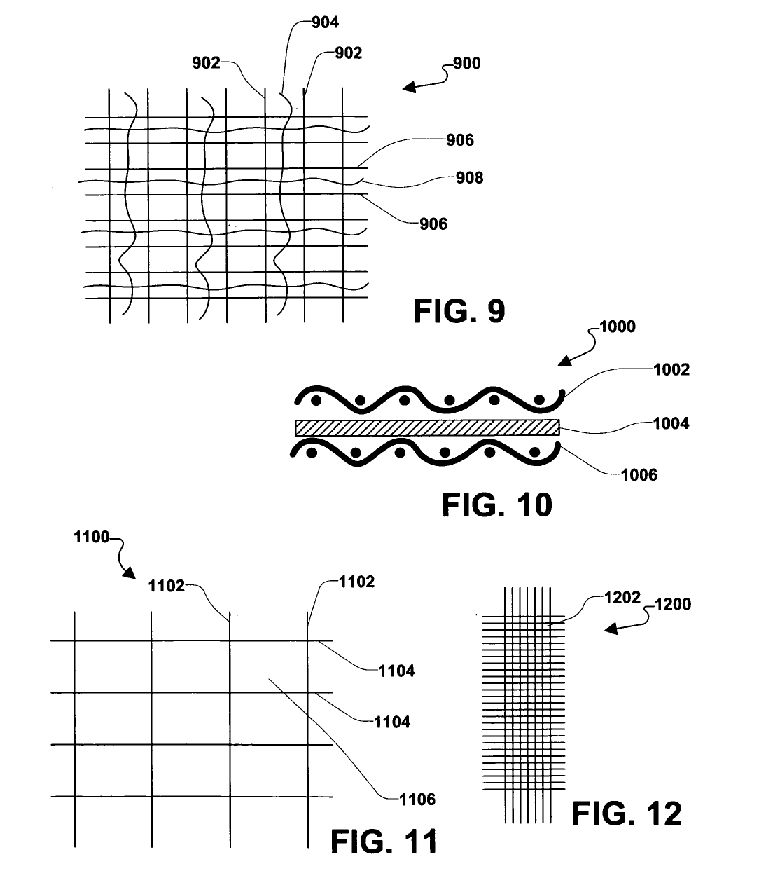

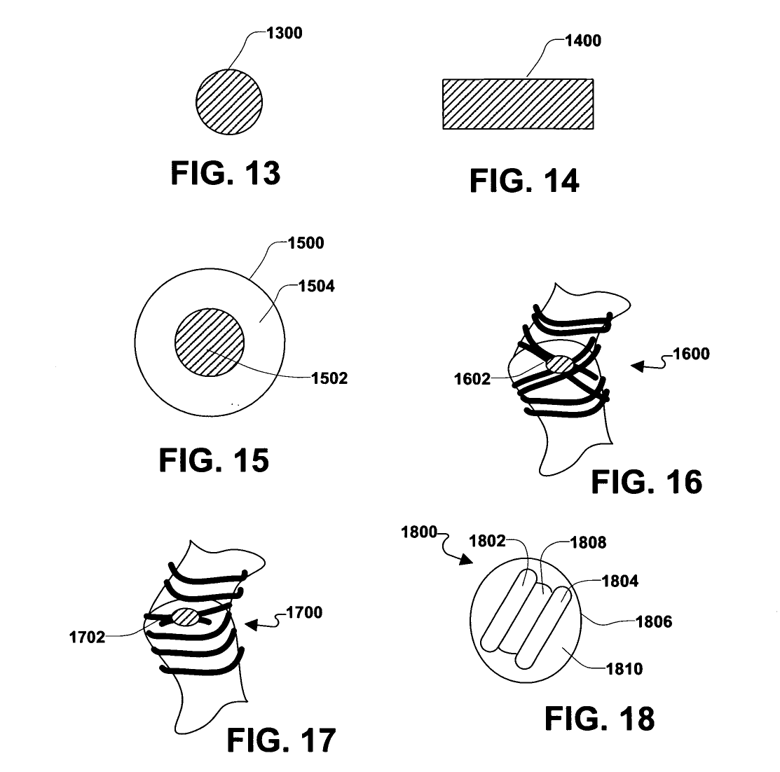

In another example, the strands can have a cross-section of one of a variety of shapes. For example, FIG. 13 includes an illustration of a strand 1300 having a circular cross-section. In another example, FIG. 14 includes an illustration of a strand 1400 having a rectangular cross-section. Alternatively, the strand can have a cross-section of a shape, such as circular, polygonal, star-shaped, Y-shaped, cross-shaped, or any variation thereon.

In a further example, the strand 1500 can include a core 1502 coated with a coating 1502, as illustrated at FIG. 15. For example, the strand 1500 can include a coating including an elastomeric material. In another example, the core 1502 can be coated with a release agent, such as a solid matrix release material or a hydrogel.

When used as a strand material, the strand can be wrapped around the zygapophysial joint. For example, the strand material can be wrapped around the processes associated with the zygapophysial joint. In an exemplary embodiment, the strand material can be wrapped multiple times around the processes forming the zygapophysial joint, such as at least about twice around the zygapophysial joint. In addition, the strand material can be wound around the zygapophysial joint in a pattern. For example, FIG. 16 illustrates a crisscross pattern 1602 around a zygapophysial joint. In another example, FIG. 17 illustrates a parallel wrap pattern 1702 around a zygapophysial joint 1700. In a particular embodiment, the pattern formed by the strand material around the zygapophysial joint can influence the relative motion of the processes of the zygapophysial joint when stressed.

### Treatment of a Patient

In an example, a healthcare provider can determine the condition of joints associated with adjacent vertebrae. For example, the healthcare provide can perform tests, such as CT scans or MRI scans, to determine the condition of intervertebral discs and zygapophysial joints of a patient. Based on the analysis, the healthcare provider can determine that a repair or support device is to be prescribed.

For example, a patient can suffer from a deterioration of zygapophysial joints between two vertebrae. In another example, the patient can have undergone an acute stress event leading to damage in capsular ligaments surrounding a zygapophysial joint or damage in the zygapophysial joint itself. In each case, a repair or support device can be prescribed. In addition to prescribing the repair or support device, the healthcare provider can prescribe other treatments such as therapeutic agents, removal and replacement of the intervertebral disc or zygapophysial joint, or fusion of the adjacent vertebra.

FIG. 18 includes an illustration of a cross-section of the zygapophysial joint 1800 surrounded by an embodiment of the repair or support device 1806. For example, the device 1806 can surround the processes 1802 and 1804 associated with the zygapophysial joint 1800. A joint tissue 1808 can be between the processes 1802 and 1804. In an alternative embodiment, the joint tissue 1808 can be replaced with a prosthetic material that can act in place of a zygapophysial joint. In another example, the joint tissue 1808 can be replaced by a gelatinous material or matrix material that promotes bone growth between the processes 1802 and 1804. In a further example, the joint tissue 1808 can be injected with a therapeutic agent prior to or after wrapping the zygapophysial joint 1800 with the device 1806.

In a particular example, the device 1806 includes a mesh material that forms a space 1810 between the device 1806 and the zygapophysial joint 1800. In an example, a gel material including therapeutic agent can be injected in the space 1810 and encased or held in place by the device 1806.

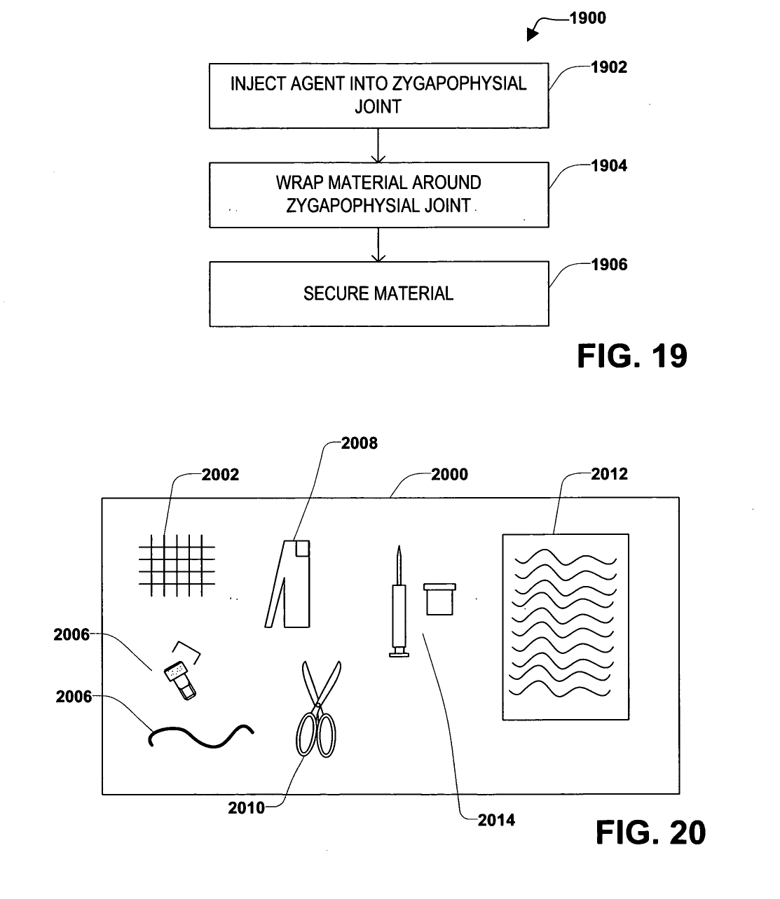

In an exemplary procedure, the zygapophysial joint can be wrapped and secured. FIG. 19 includes a flow diagram of an exemplary method 1900. For example, once a patient has been prepared for surgery and access to the zygapophysial joint is acquired, the zygapophysial joint optionally can be injected with a therapeutic agent, as illustrated at 1902. Alternatively, the zygapophysial joint can be removed and replaced with a prosthetic device or a fusion device.

The device including the mesh material or the strand material can be wrapped around the zygapophysial joint, as illustrated at 1904. In particular, the mesh material can be wrapped around the zygapophysial joint and capsular ligaments. In another example, the strand material can be wrapped around the zygapophysial joint to engage the articular processes associated with the zygapophysial joint. In a particular example, the strand material can be wrapped around the zygapophysial joint, not passing through the zygapophysial joint or the articular processes associated with the zygapophysial joint.

The device can be secured to engage the zygapophysial joint, as illustrated at 1906. For example, the mesh material can be secured to the processes associated with the zygapophysial joint. In a particular example, the mesh material can be secured to the superior articular process of the inferior vertebra and can be secured to the inferior articular process of the superior vertebra. Alternatively, the mesh material can be secured to itself. In an example, the mesh material can be secured using a fastener. For example, the fastener can include a staple. In another example, the fastener can include a screw. In a further example, the fastener can include an adhesive such as a tissue adhesive or a bone cement. In an additional example the fastener can include a strand material surrounding a first articular process and secured to itself and can include a strand material surrounding a second articular process and secured to itself. In a further example, the fastener can include a crimp joint or connector to secure the mesh to itself.

In another exemplary embodiment, the strand material absent the mesh material can be wrapped around the processes associated with the zygapophysial joint and secured to itself. For example, the strand material can be inserted into a fastener and crimped to itself. In another example, the fastener can engage ends of the strand material, securing them together.

In a further exemplary embodiment, therapeutic agent can be injected into the joint, into the space between the joint and the device, or into the device once the device is installed.

### Treatment Kit

The mesh material or the strand material used to form the device can be included in a kit. In an exemplary embodiment, FIG. 20 includes an illustration of an exemplary kit 2000. For example, the kit can include a mesh material 2002. The mesh material 2002 can be adapted to engage a zygapophysial joint. In addition or alternatively, the kit 2000 can include a strand material 2004 adapted to engage a zygapophysial joint.

In addition, the kit 2000 can include a tool to further adapt the mesh material 2002 or the strand material 2004, such as scissors 2010. For example the mesh material 2002 or the strand material 2004 can be cut based on the location or the size of the zygapophysial joint.

In another example, the kit 2000 can include one or more fasteners 2006. For example, the kit 2000 can include staples, screws, or crimp fasteners to secure the mesh material 2002 or the strand material 2004. In a further example, the kit 2000 can include a tool 2008 to secure the mesh material 2002 or the strand material 2004. For example, the tool 2008 can be a stapler or a screwdriver to secure the mesh material 2002 to a process associated with the zygapophysial joint. In another example, the tool 2008 can include a crimp tool to secure the strand material 2004 or the mesh material 2002 to itself.

In an additional example, the kit 2000 can include a therapeutic agent 2014. For example, the kit 2000 can include a therapeutic agent 2014 and a syringe for injecting the therapeutic agent 2014 into the zygapophysial joint. In another example, the syringe can include a gel that includes the therapeutic agent 2014 for injection into a space between the mesh material 2002 and the zygapophysial joint. In an alternative embodiment, the syringe can include an adhesive, gel material, or bone cement to facilitate fusion of the zygapophysial joint.

In a particular embodiment, the kit 2000 includes an indication of the use of the mesh material 2002 or strand material 2004. For example, an indicator 2012 can identify the kit 2000 as a zygapophysial joint repair or support system. In another example, the indicator 2012 can include contraindications for use of the kit 2000 and materials 2002 and 2004. In a further example, the indicator 2012 can include instructions, such as instructions regarding the installation of the device and materials 2002 and 2004. In particular, the instructions can include elements illustrated in FIG. 19.

In an exemplary embodiment, the kit components can be disposed in a closed container, which can be adequate to maintain the contents of the container therein during routine handling or transport, such as to a healthcare facility or the like.

### CONCLUSION

With embodiments of the device described above, the condition of a zygapophysial joint can be maintained, repaired, or secured. Such a device can be used to limit further deterioration of a degrading zygapophysial joint. In another example, such a device can be used to secure the zygapophysial joint during fusion of the associated articular processes. In an additional example, the device can be used to permit healing of capsular ligaments or the zygapophysial joint after an acute stress injury.

In a particular embodiment, the device can act to limit undesired movement of the processes and the associated vertebra relative to each other. As such, the device can reduce the likelihood of further injury to a zygapophysial joint, reduce pain associated with zygapophysial joint damage, and complement other devices, such as implants and fusion devices.

The above-disclosed subject matter is to be considered illustrative, and not restrictive, and the appended claims are intended to cover all such modifications, enhancements, and other embodiments that fall within the true scope of the present invention. For example, it is noted that the configuration of devices in the exemplary embodiments described herein can have alternative configurations. Thus, to the maximum extent allowed by law, the scope of the present invention is to be determined by the broadest permissible interpretation of the following claims and their equivalents, and shall not be restricted or limited by the foregoing detailed description.

1. A kit comprising:
a mesh material adapted to engage a zygapophysial joint; and
an indicator associating the kit with treatment of the zygapophysial joint.

2. The kit of claim 1, wherein the mesh material includes a therapeutic agent absorbed therein.

3. The kit of claim 2, wherein the mesh material includes strands of release material interwoven in the mesh material, the strands including the therapeutic agent.

4. The kit of claim 2, wherein the mesh material includes a release material adjacent to a sheet of woven material, the release material including the therapeutic agent.

5. The kit of claim 1, wherein the mesh material includes a warp strand and a weft strand, wherein the warp strand and the weft strand intersect to form an acute angle.

6. The kit of claim 5, wherein the acute angle is not greater than about 65°.

7. The kit of claim 1, wherein the mesh material includes an elastomeric coating.

8. The kit of claim 1, wherein the mesh material has an open area of at least about 75%.

9. The kit of claim 1, wherein the mesh material has an open area of not greater than about 20%.

10. (canceled)

11. The kit of claim 1, further comprising a fastener adapted to couple the mesh material to an articular process.

12. The kit of claim 11, wherein the fastener includes a staple.

13. The kit of claim 11, wherein the fastener includes a screw.

14. The kit of claim 11, wherein the fastener includes a tether.

15. The kit of claim 1, further comprising a fastener adapted to couple the mesh to itself.

16. The kit of claim 1, wherein the indicator includes a contraindication.

17. The kit of claim 1, wherein the indicator includes instructions for use.

18. (canceled)

19. The kit of claim 1, further comprising a tool.

20. (canceled)

21. (canceled)

22. (canceled)

23. A device comprising:
a mesh material adapted to engage a zygapophysial joint; and
a fastener configured to secure the mesh material.

24. The device of claim 23, wherein the fastener is configured to secure the mesh to an articular process associated with the zygapophysial joint.

25. The device of claim 23, wherein the fastener is configured to secure the mesh to itself.

26. The device of claim 23, wherein the mesh material includes a therapeutic agent absorbed therein.

27. The device of claim 26, wherein the mesh material includes strands of release material interwoven in the mesh material, the strands including the therapeutic agent.

28. The device of claim 26, wherein the mesh material includes a release material adjacent to a sheet of woven material, the release material including the therapeutic agent.

29. The device of claim 23, wherein the mesh material includes a warp strand and a weft strand, wherein the warp strand and the weft strand intersect to form an acute angle.

30. (canceled)

31. The device of claim 23, wherein the mesh material includes an elastomeric coating.

32. The device of claim 23, wherein the mesh material has an open area of at least about 75%.

33. The device of claim 23, wherein the mesh material has an open area of not greater than about 20%.

34. (canceled)

35. (canceled)

36. (canceled)

37. (canceled)

38. A kit comprising:
a strand material configured to engage a zygapophysial joint; and
instructions indicating that the strand material is to be encircled around two articular processes associated with the zygapophysial joint.

39. The kit of claim 38, wherein the instructions indicate that the strand material is to be encircled around the two articular processes of the zygapophysial joint at least twice.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)
